(19) United States
(12) Patent Application Publication
(10) Pub. No.: US 2024/0117405 A1
(43) Pub. Date: Apr. 11, 2024

(54) COLORIMETRIC BIOSENSOR, PREPARATION METHOD THEREOF, AND ANTIBIOTIC SUSCEPTIBILITY TESTING METHOD USING THE SAME

(71) Applicant: INHA UNIVERSITY RESEARCH AND BUSINESS FOUNDATION, Incheon (KR)

(72) Inventors: Tae Joon Jeon, Gyeonggi-do (KR); Sun Min Kim, Seoul (KR); Hui Soo Jang, Incheon (KR); Woo Jin Jeong, Gyeonggi-do (KR); Seo Yoon Song, Seoul (KR)

(21) Appl. No.: 18/284,252

(22) PCT Filed: Dec. 21, 2022
(86) PCT No.: PCT/KR22/20936

§ 371 Date: Sep. 26, 2023

(30) Foreign Application Priority Data
Jan. 7, 2022 (KR) 10-2022-0002636

(51) Int. Cl. C12Q 1/18; G01N 21/78

(52) U.S. Cl. CPC C12Q 1/18; G01N 21/78

(57) ABSTRACT

The present invention relates to a colorimetric biosensor, preparation method thereof, and antibiotic susceptibility testing method using the same, and more specifically, in the present invention, it is possible to prepare a colorimetric biosensor comprising a porous hydrogel structure including polydiacetylene and a hydrogel polymer (alginate, PEG-DA, etc.); and a microbial nutrient source, and it may be applied to a colorimetric biosensor for detecting microorganisms or a method for testing antibiotic susceptibility of microorganisms for allowing in real-time measurement and exhibiting excellent sensitivity using the colorimetric biosensor.

### TECHNICAL FIELD

The present invention relates to a colorimetric biosensor, preparation method thereof, and antibiotic susceptibility testing method using the same.

### BACKGROUND ART

A colorimetric biosensor is a material or device that may provide easier and useful information for data analysis by detecting signals using the characteristics of colorimetric or fluorescent nanomaterials at the cellular level or in vivo level.

As a sensor material of such a biosensor, polydiacetylene (PDA) has the advantage of being easily synthesized in an aqueous solution and the merit as a sensor material because most biologically important target materials such as DNA, proteins, carbohydrates, and ions are hydrophilic. Further, it is known that polydiacetylene does not require the use of an additional catalyst or initiator and changes color to blue by UV stimulation, but changes color to red by external physical, chemical, and biological stimulation.

Meanwhile, a test for selecting antibiotics capable of inhibiting the growth of microorganisms is called an antibiotic susceptibility test, which is a direct and important test that allows selection of the type of antibiotic to be used for microorganisms. Further, when prescribing an appropriate antibiotic for a patient, it allows customized prescription by considering the prescription method, number of times, cost, and side effects. When the susceptibility results are used, the increase in treatment cost and the disappointment of the guardians that may occur when antibiotics are prescribed empirically may be reduced, and it provides an opportunity to reduce acquisition of bacterial resistance and complications and shorten the patient's recovery period.

The most common antibiotic susceptibility tests are the disk diffusion technique and the broth dilution technique. However, these methods require a process of culturing the bacteria for several days, identifying the bacteria, and then measuring the turbidity, and this process takes a long time and requires a lot of labor.

Therefore, it is necessary to develop a method capable of real-time measurement, reducing time and labor, and overcoming the problems of conventional antibiotic susceptibility testing methods.

Accordingly, the present inventors have prepared a colorimetric biosensor including a porous hydrogel structure including polydiacetylene and a hydrogel polymer (alginate, PEG-DA, etc.); and a microbial nutrient source, and have completed the present invention in view of the fact that this may be applied to colorimetric biosensor or antibiotic susceptibility test method of microorganisms for detecting microorganisms in real-time in and exhibiting excellent sensitivity.

### DISCLOSURE

### Technical Problem

The present invention has been derived in consideration of the above problems, and an object of the present invention is to prepare a colorimetric biosensor including a porous hydrogel structure including polydiacetylene and a hydrogel polymer (alginate, PEG-DA, etc.); and a microbial nutrient source, and it may be applied to a method for testing antibiotic susceptibility of microorganisms for allowing in real-time measurement and exhibiting excellent sensitivity using the colorimetric biosensor.

Problems to be solved by the present invention are not limited to the problems mentioned above, and other problems not mentioned are clearly understood by those skilled in the art from the description below.

### Technical Solution

In order to achieve the above purpose, the present invention provides a colorimetric biosensor comprising a porous hydrogel structure formed of a hydrogel polymer and polydiacetylene; and a microbial nutrient source.

The microbial nutrient source may be encapsulated inside the porous hydrogel structure.

The microbial nutrient source may be in the form of a shell surrounding the surface of the porous hydrogel structure.

The hydrogel polymer may include at least one kind selected from the group consisting of alginate, agarose, chitosan, poly (ethylene glycol) diacrylate (PEG-DA), hydroxyethylene methacrylate (HEMA), polyvinyl alcohol (PVA) and polyacrylamide (PAM).

The microbial nutrient source may include at least one selected from the group consisting of Luria-Berani (LB) broth, LB agar broth, tryptic soy broth (TSB), lactobacilli MRS broth, R2A broth (MB-R2230), Mueller Hinton (M-H) broth, and a broth containing casein hydrolysate (casamino acid).

The colorimetric biosensor may detect a microorganism through color change.

The microorganism may include at least one selected from the group consisting of isolated cells of bacteria, fungus, alga, protozoan and metazoan.

Further, the present invention provides a method for testing susceptibility to an antibiotic, including culturing a microorganism in the presence of the colorimetric biosensor according to the present invention and the antibiotic.

It may be determined that the microorganism has susceptibility to the antibiotic when there is no colorimetric change of the colorimetric biosensor, and it may be determined that the microorganism has resistance to the antibiotic when there is colorimetric change of the colorimetric biosensor.

Further, the present invention provides a kit for testing susceptibility to an antibiotic, the kit including the colorimetric biosensor according to the present invention and the antibiotic.

Further, the present invention provides a method for preparing a colorimetric biosensor, the method including a step (a) of obtaining a hydrogel precursor solution containing a polydiacetylene solution, a hydrogel polymer and a microbial nutrient source, a step (b) of dropping the hydrogel precursor solution into a calcium chloride solution to obtain a hydrogel structure, and a step (c) of irradiating ultraviolet rays having 200 to 300 nm wavelength to the hydrogel structure.

The concentration of the hydrogel polymer in the hydrogel precursor solution may be 1.0 to 15.0% (w/v).

The concentration of the polydiacetylene solution may be 0.5 to 5 mM.

The concentration of the calcium chloride solution may be 0.5 to 20.0% (w/v).

The colorimetric biosensor may include a porous hydrogel structure formed of a hydrogel polymer and polydiacetylene and a microbial nutrient source, and the microbial nutrient source may be encapsulated inside the porous hydrogel structure, or the microbial nutrient source may be in the form of a shell surrounding the surface of the porous hydrogel structure.

### Advantageous Effects

According to the present invention, a colorimetric biosensor including a porous hydrogel structure including polydiacetylene and a hydrogel polymer and a microbial nutrient source may be prepared, and it may be applied to a method for testing antibiotic susceptibility of microorganisms for allowing in real-time measurement and exhibiting excellent sensitivity using the colorimetric biosensor.

It should be understood that the effects of the present invention are not limited to the above effects and includes all effects that may be inferred from the detailed description of the present invention, or the configuration of the invention described in the claims.

### DESCRIPTION OF DRAWINGS

FIG. 1 is an image showing a process of detecting microorganisms through a colorimetric biosensor according to an example of the present invention [Colorimetric biosensor: blue before incubation with bacteria->red after incubation with microorganisms].

FIG. 2 is an image showing a process of preparing a colorimetric biosensor of (a) layered form and (b) encapsulated form according to an example of the present invention [PDA-alginate beads: blue, LB: yellow, PDA-alginate-LB beads: blue].

FIG. 3 is an image showing a colorimetric biosensor of (a) layered form and (b) encapsulated form prepared according to an example of the present invention.

FIG. 4 is an image of the experimental results showing the difference in sensitivity when the LB broth is located (a) outside and (b) inside the encapsulated colorimetric biosensor [T1: 0 h, T2: 6 h, T3: 12 h, T4: 18 h and T5: 24 h].

FIG. 5 is an image of an antibiotic susceptibility test result using a colorimetric biosensor in an encapsulated form.

### MODES OF THE INVENTION

Hereinafter, examples of the present invention are described in more detail with reference to the accompanying drawings. Examples of the present invention may be modified in various forms, and the scope of the present invention should not be construed as being limited to the following examples. The present examples are provided to more completely explain the present invention to those skilled in the art. Accordingly, the shapes of elements in the drawings are exaggerated to emphasize clearer description.

Conventionally, a disk diffusion technique and a broth dilution technique have been commonly performed for antibiotic susceptibility testing. However, these methods require a process of culturing the bacteria for several days, identifying the bacteria, and then measuring the turbidity, and this process takes a long time and requires a lot of labor.

Therefore, it is necessary to develop a method capable of real-time measurement, reducing time and labor, and overcoming the problems of conventional antibiotic susceptibility testing methods.

In order to solve the above problem, the present invention prepares a colorimetric biosensor including a porous hydrogel structure including polydiacetylene and a hydrogel polymer and a microbial nutrient source, and it may be applied to a method for testing antibiotic susceptibility of microorganisms for allowing in real-time measurement and exhibiting excellent sensitivity using the colorimetric biosensor.

Colorimetric Biosensor

The present invention provides a colorimetric biosensor comprising a porous hydrogel structure formed of a hydrogel polymer and polydiacetylene (PDA); and a microbial nutrient source.

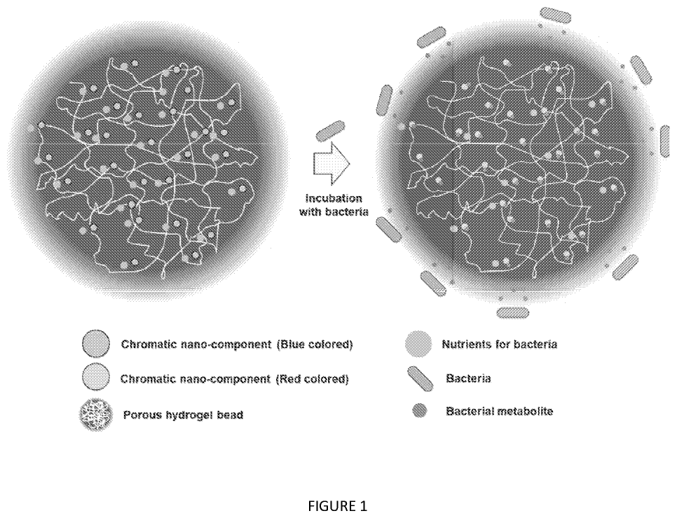

According to one example of the present invention, the colorimetric biosensor may detect a microorganism through color change. Specifically, when the colorimetric biosensor is cultured with a microorganism, the nutrient source of the microorganism contained in (inside) the sensor diffuses to the outside, and the microorganism uses it as a growth source to proliferate. Biomolecules, which are metabolites produced by the growth of these microorganisms, penetrate into the hydrogel through the pores of the porous hydrogel structure, stimulate the PDA, and induce color change of the colorimetric biosensor. Referring to FIG. 1, it may be confirmed that the colorimetric biosensor has a blue color before incubation with bacteria, and then changes color to red by the aforementioned color change mechanism after incubation with bacteria.

More specifically, microorganisms, particularly bacteria, have a characteristic of recognizing growth sources in the surrounding environment and growing. That is, bacteria may grow while gathering in the direction where the growth source is located. For use of these characteristics in the present invention, a nutrient source that may act as a growth source and PDA are included in the hydrogel, so that bacteria may grow as close as possible to the sensor material PDA.

Further, the PDA is characterized by a change in its internal structure due to various stimuli (temperature, pH, electrical/physical stimulation, etc.) occurring on the surface, and accordingly a color change from blue to red. Accordingly, some of the metabolites produced by the bacteria interact with the surface of the PDA, thereby causing a color change by stimulating it.

Such color change may be confirmed with the naked eye and may be analyzed numerically through an image processing device. Therefore, the presence and proliferation rate of microorganisms may be measured through color change.

More specifically, the colorimetric biosensor may measure the presence and growth rate of microorganisms through a color transition from blue to red. The colorimetric biosensor may have a blue color and as described above, may exhibit a red color transition phenomenon due to biomolecules produced from microorganisms through an incubation process with microorganisms.

According to one example of the present invention, the hydrogel polymer may include at least one selected from the group consisting of alginate, agarose, chitosan, poly (ethylene glycol) diacrylate (PEG-DA), hydroxyethylene methacrylate (HEMA), polyvinyl alcohol (PVA) and polyacrylamide (PAM), but is not limited thereto. Specifically, the hydrogel polymer may be alginate, and in this case, it may exhibit excellent color change sensitivity.

According to one example of the present invention, the porous hydrogel structure may have a spherical shape, but is not limited thereto. Specifically, it may be in a bead shape.

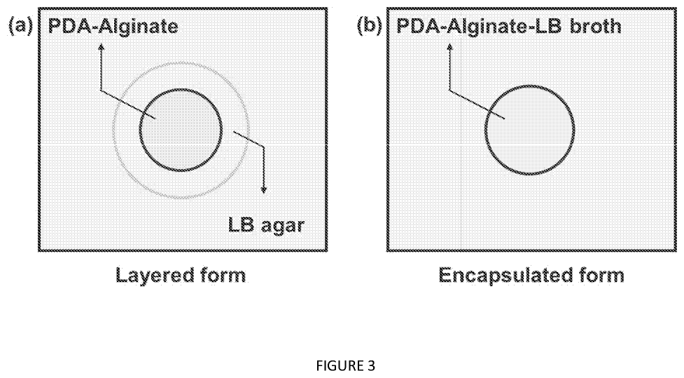

According to one example of the present invention, the microbial nutrient source may be in the form of a shell surrounding the surface of the porous hydrogel structure (See FIG. 3A). In this case, it is easier for the microorganisms to access the nutrient source of the microorganisms, thereby improving the sensitivity to the microorganisms.

According to one example of the present invention, the microbial nutrient source may be encapsulated inside the porous hydrogel structure (See FIG. 3B). In this case, it is confirmed that, in particular, the sensitivity to microorganisms is more improved than when the microbial nutrient source surrounds the surface of the structure.

The microbial nutrient source may include at least one selected from the group consisting of Luria-Berani (LB) broth, LB agar broth, tryptic soy broth (TSB), lactobacilli MRS broth, R2A broth (MB-R2230), Mueller Hinton (M-H) broth, and a broth containing casein hydrolysate (casamino acid). Specifically, the microbial nutrient source may be LB broth. More specifically, the microbial nutrient source may be an LB broth medium composed of tryptone, yeast extract, and NaCl, or an LB agar medium in which tryptone, yeast extract, NaCl, and agar are mixed, but is not limited thereto. The microbial nutrient source is not limited to the above types, and anyone may be used as long as it may be used as a nutrient source for target microorganisms to be detected.

The microorganism may include at least one selected from the group consisting of isolated cells of bacteria, fungus, alga, protozoan and metazoan, but is not limited thereto. Specifically, the microorganism may be a bacterium. More specifically, the microorganisms may be a bacterium of to the genus Escherichia and Pseudomonas, which are representative Gram-negative bacteria and may be a bacterium of the genus Staphylococcus or Enterococcus, which are representative Gram-positive bacteria.

According to one example of the present invention, incubation according to the present invention may be performed in sterile water. Since the broth components necessary for culturing microorganisms are contained in the nutrient source according to the present invention and exist inside or outside the porous hydrogel structure, another medium is unnecessary for microorganism incubation, and incubation may be performed in sterile water, but is not limited thereto.

The incubation is performed to obtain the proliferation of microorganisms to be detected. Techniques and conditions for incubating microorganisms are particularly well known to those skilled in the art, who are aware of the appropriate nutrient broth, the optimum growth temperature, for example, 37° C. for many bacteria pathogenic to mammals, and the required conditions, for the respective microorganisms, for the growth of the microorganisms according to the present invention. The incubation time varies for each microorganism depending on the growth rate and generation time, i.e., the time required for the microorganism to divide into two progeny microorganisms. Generally, the incubation time is less than 72 hours, 48 hours or 24 hours. Further, the incubation is stopped as soon as the information sought, i.e., detection, quantification, or susceptibility to antibiotics is obtained.

Use of Colorimetric Biosensor

The present invention provides a method for testing susceptibility to an antibiotic, including culturing a microorganism in the presence of the colorimetric biosensor according to the present invention and the antibiotic.

The antibiotic susceptibility is also referred to as antibiotic sensitivity and means that the growth inhibition of the microorganism (strain, etc.) is affected by a specific antibiotic, and according to the conventional antibiotic susceptibility test method, in case of treating with antibiotics, when bacteria do not grow around the area treated with antibiotics, it is said to have susceptibility. Antibiotic susceptibility test results may be divided into, for example, susceptibility, intermediate susceptibility (intermediate resistance) and resistance. In order to treat infections caused by microorganisms, susceptible antibiotics must be used, the infection of the strain read as susceptible may be meant to be treated with the recommended dose of the antimicrobial agent for the infection of the strain and the site, intermediate susceptibility (or intermediate resistant) means that the minimum inhibitory concentration of the antimicrobial agent for the test strain is similar to the blood or tissue concentration, and therefore the therapeutic effect is lower than that for the susceptible strain, and there is a therapeutic effect when infection occurs in areas where antibacterial agents such as urine are concentrated or when the maximum amount of a drug that may be administered in large quantities is administered, and resistant means not cured by blood levels at normal doses.

According to one example of the present invention, it may be determined that the microorganism has susceptibility to the antibiotic when there is no colorimetric change of the colorimetric biosensor, and it may be determined that the microorganism has resistance to the antibiotic when there is colorimetric change of the colorimetric biosensor.

Specifically, as described above, when the colorimetric biosensor is cultured with a microorganism, the nutrient source of the microorganism contained in (inside) the sensor moves to the outside, and the microorganism uses it as a growth source to proliferate. Biomolecules, which are metabolites produced by the growth of these microorganisms, penetrate into the hydrogel through the pores of the porous hydrogel structure, stimulate the PDA, and induce color change of the colorimetric biosensor.

At this time, when antibiotics are present around the biosensor, microorganisms showing susceptibility to the antibiotics cease to proliferate and do not produce biomolecules as metabolites, and as a result, colorimetric change of the colorimetric biosensor may not be observed. On the other hand, microorganisms showing resistance to the antibiotic proliferate and produce biomolecules as metabolites, and as a result, a colorimetric change of the colorimetric biosensor may be observed.

As such, it is possible to provide a method for testing antibiotic susceptibility of microorganisms capable of real-time measurement and excellent sensitivity using the colorimetric biosensor according to the present invention.

According to one example of the present invention, the antibiotic may include at least one selected from the group consisting of penicillin-based antibiotics, glycopeptide-based antibiotics, quinolone-based antibiotics, cephalosporin-based antibiotics, tetracycline-based antibiotics, sulfonamide-based antibiotics, polyether-based antibiotics, and peptide-based antibiotics, but is not limited thereto. Specifically, the antibiotics may be penicillin-based and glycopeptide-based antibiotics.

Further, the present invention provides a kit for testing susceptibility to an antibiotic, the kit including the colorimetric biosensor according to the present invention and the antibiotic. Specifically, the colorimetric biosensor may be provided as a conventional kit capable of confirming the susceptibility or resistance of a target material (microorganism, etc.) to a specific antibiotic.

As a specific example, the kit for testing susceptibility to antibiotic may include a well plate to which the colorimetric biosensor is added or treated and an antibiotic, respectively, or a well plate to which the colorimetric biosensor and antibiotic are added or treated together. In this case, the antibiotics are contained in different concentrations in each well constituting the kit, and for example, a medium containing various antibiotic to be tested at different concentrations, such as 0.25, 0.5, 1, 2, 4, 8, 16, or 32 μg/mL, is put into each well constituting the kit. The amount of the broth varies depending on the volume of individual wells constituting the multi-well plate, but in general, an average of 200 μl may be added to each well but is not limited thereto.

Sterile water may be further included in each well constituting the kit. As described above, since the broth components necessary for culturing microorganisms are contained in the nutrient source according to the present invention and exist inside or outside the porous hydrogel structure, another medium is unnecessary for microorganism incubation, and incubation may be performed in sterile water, but is not limited thereto.

Method for Preparing Colorimetric Biosensor

The present invention provides a method for preparing a colorimetric biosensor, the method including a step (a) of obtaining a hydrogel precursor solution containing a polydiacetylene solution, a hydrogel polymer and a microbial nutrient source, a step (b) of dropping the hydrogel precursor solution into a calcium chloride solution to obtain a hydrogel structure, and a step (c) of irradiating ultraviolet rays having 200 to 300 nm wavelength to the hydrogel structure.

According to one example of the present invention, step (a) is a step of preparing a hydrogel precursor solution for forming a hydrogel structure, and may be performed by mixing a PDA solution, a hydrogel polymer, and a microbial nutrient source.

The concentration of the hydrogel polymer in the hydrogel precursor solution may be 1.0 to 15.0% (w/v), 5.0 to 13.0% (w/v) or 8.0 to 10.0% (w/v). When the concentration of the hydrogel polymer is 1.0% (w/v) or more, the ability to form a gel is excellent, and when it is 15.0% (w/v) or less, the ability to preserve the shape of a spherical porous hydrogel structure is excellent, and the viscosity is appropriate, so it is easy to handle. Specifically, when the concentration range of the hydrogel polymer is maintained, the above critical effect may be maintained even if the mixing ratio of the PDA solution and the microbial nutrient source is variously adjusted.

The concentration of the PDA solution may be 0.5 to 5 mM, 0.5 to 4 mM, or 1.0 to 3 mM. The concentration of the PDA solution may affect the process of obtaining the hydrogel structure in step (b), and the formation of the hydrogel structure is easy in the concentration range of the PDA solution.

The concentration of the calcium chloride solution may be 0.5 to 20.0% (w/v), 0.5 to 10.0% (w/v), 0.5 to 5.0% (w/v) or 0.5 to 1.5% (w/v). In the concentration range of the calcium chloride solution, there is an effect of further improving the sensitivity of the colorimetric biosensor produced.

According to one example of the present invention, step (b) is a step of obtaining a gelled hydrogel structure through the reaction of the hydrogel precursor solution with the calcium chloride, and it may simultaneously perform a process of stirring the calcium chloride solution using a magnetic bar so that the dropped droplets do not agglomerate.

According to one example of the present invention, after step (b), a process of washing the hydrogel structure in which the gelation is completed with distilled water may be further performed.

According to one example of the present invention, step (c) may be a step of obtaining a blue colorimetric biosensor by irradiating ultraviolet rays of 200 to 300 nm, 230 to 280 nm, 240 to 270 nm or 250 to 260 nm wavelength to the hydrogel structure. Ultraviolet rays in the above wavelength range are irradiated to easily perform color transition to blue.

The colorimetric biosensor may include a porous hydrogel structure formed of a hydrogel polymer and polydiacetylene and a microbial nutrient source, and the microbial nutrient source may be encapsulated inside the porous hydrogel structure, or the microbial nutrient source may be in the form of a shell surrounding the surface of the porous hydrogel structure, but its formation is not limited thereto.

Descriptions mentioned in the colorimetric biosensor of the present invention, its use, and its manufacturing method are equally applied unless they contradict each other.

The above description is a description of the technical idea of the present invention using an example, and various modifications and variations may be made to those skilled in the art without departing from the essential characteristics of the present invention. Therefore, the examples described in the present invention are not intended to limit the technical spirit of the present invention, but to explain, and the scope of the technical spirit of the present invention is not limited by these examples. The protection scope of the present invention should be construed according to the claims, and all technical ideas within the equivalent scope thereof should be construed as being included in the scope of the present invention.

Hereinafter, the present invention will be described in more detail through examples.

### EXAMPLE

### Example 1. Preparation of Colorimetric Biosensor in Encapsulated Form

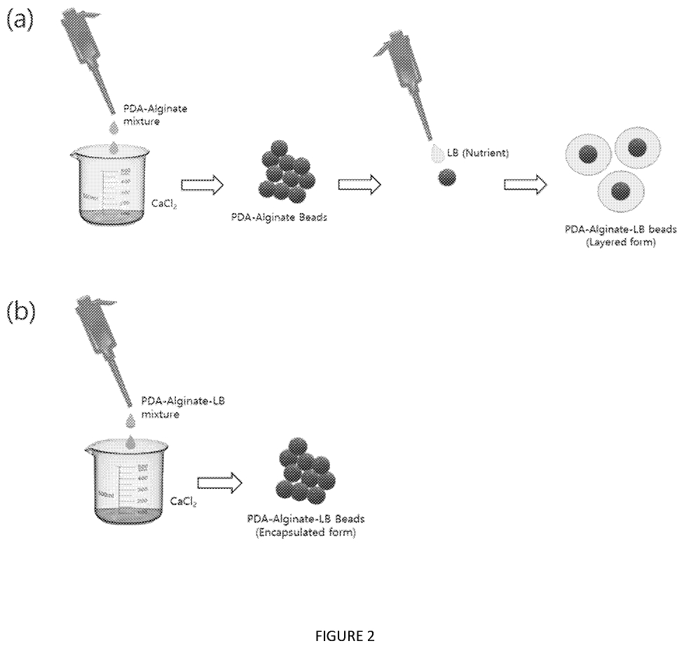

A hydrogel precursor solution was prepared by mixing a 3 mM polydiacetylene (PDA) solution, alginate (9% (w/v) compared to the hydrogel precursor solution), and LB broth. The hydrogel precursor solution was dripped into a beaker filled with 1% (w/v) calcium chloride (CaCl₂)) solution through a syringe needle. The calcium chloride solution was continuously stirred using a magnetic bar so that the dripped droplets do not agglomerate, thereby reacting the hydrogel precursor solution with calcium chloride to form a gel (gelation). The gelated hydrogel structure was taken out of the calcium chloride solution, washed with distilled water, and then irradiated with 254 nm wavelength UV to convert the color of the PDA inside the hydrogel structure to blue to prepare an encapsulated colorimetric biosensor (See FIGS. 2B and 3B).

### Example 2. Preparation of Colorimetric Biosensor in Layered Form

A hydrogel precursor solution was prepared by mixing a 3 mM polydiacetylene (PDA) solution and alginate (9% (w/v) compared to the hydrogel precursor solution). The hydrogel precursor solution was dripped into a beaker filled with 1% (w/v) calcium chloride (CaCl₂)) solution through a syringe needle. The calcium chloride solution was continuously stirred using a magnetic bar so that the dripped droplets do not agglomerate, thereby reacting the hydrogel precursor solution with calcium chloride to form a gel (gelation). The gelated hydrogel structure was taken out of the calcium chloride solution, washed with distilled water, and then irradiated with 254 nm wavelength UV to convert the color of the PDA inside the hydrogel structure to blue. Thereafter, the gelated hydrogel structure was dropped onto a hydrophobic liquid LB agar broth using a pipette to cover it in the form of a thin film. The coated LB agar medium hardened immediately due to the low temperature, finally preparing a colorimetric biosensor having a bacterial growth source in a layered form (See FIGS. 2A and 3A).

### Experimental Example 1. Susceptibility Sensitivity Comparison Experiment

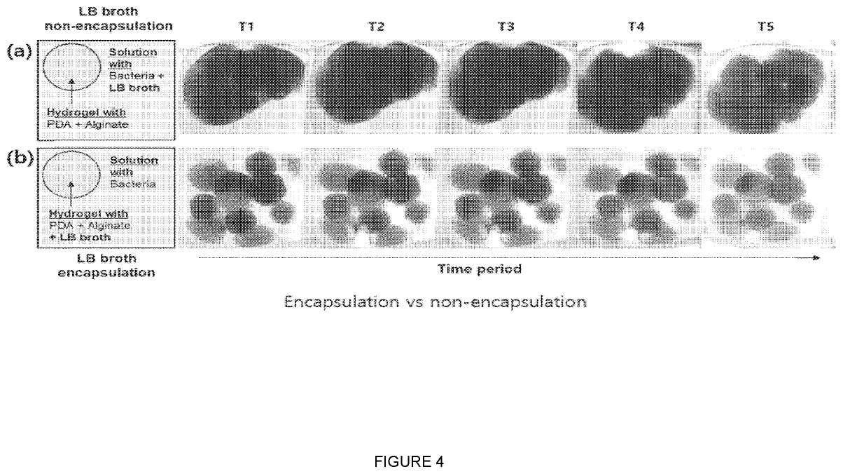

When the LB broth was located (a) outside and (b) inside the encapsulated colorimetric biosensor, a susceptibility sensitivity comparison experiment was performed and shown in FIG. 4.

Specifically, when preparing the colorimetric biosensor in an encapsulated form from Example 1, it was prepared in a form in which the LB broth was not contained, or the LB broth was contained. The well containing the biosensor without LB broth was filled with 10⁸ CFU/mL of E. coli and LB broth (See FIG. 4A). The well containing the biosensor with LB broth was filled with only 10⁸ CFU/mL of E. coli (See FIG. 4B). To establish identical experimental conditions, LB broth was prepared with the same concentration. In order to induce the growth of E. coli and the color change of the biosensor accordingly, they were cultured at a temperature of 37° C., and each well plate was photographed at 0, 6, 12, 18 and 24 hours to compare the color change.

Referring to FIG. 4, it may be confirmed that the color change of the PDA occurred faster in the case where the LB broth was included in the biosensor (See FIG. 4B) than in the case where it was not included (See FIG. 4A). Specifically, referring to FIG. 4B, when the LB broth was located inside the colorimetric biosensor, it showed a color change (blue->red) after 6 hours (T2), but referring to FIG. 4A, when the LB broth was located outside the colorimetric biosensor, it showed a color change (blue->red) after 18 hours (T4). Therefore, it may be determined that E. coli recognizes the LB broth inside the biosensor, grows on the surface of the biosensor, and the biomolecule released accordingly moves more easily into the inside of the biosensor, leading to faster PDA color change.

### Experimental Example 2. Antibiotic Susceptibility Test

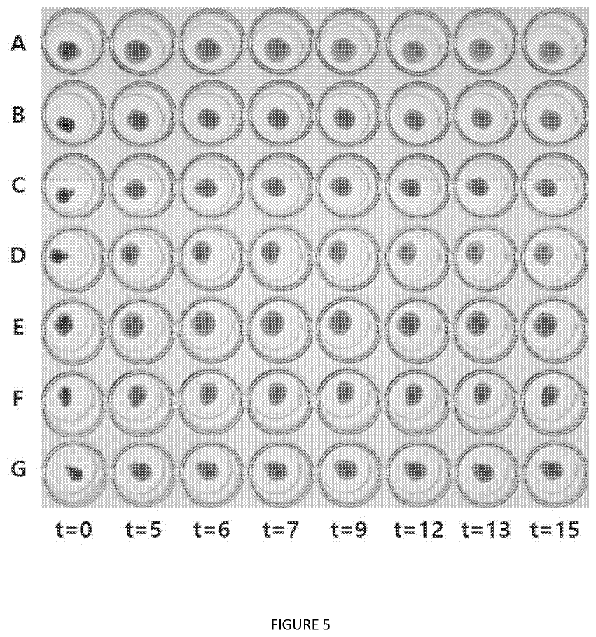

An antibiotic susceptibility test was performed on the colorimetric biosensor in encapsulated form prepared in Example 1, and the results are shown in FIG. 5.

Specifically, an encapsulated colorimetric biosensor was prepared from Example 1 and introduced into wells containing 2 and 4 μg/mL of ampicillin antibiotics (See FIGS. 5B and 5C) and 0.25, 2 and 4 μg/mL of vancomycin antibiotics (See FIGS. 5D, 5E, and 5F). Further, for the antibiotic susceptibility test, methicillin-resistant Staphylococcus Aureus (MRSA) bacteria, which are resistant to ampicillin and susceptible to vancomycin, were injected into each well at an amount of 10⁶ CFU/mL. For comparative experiments, wells containing only the colorimetric biosensor and bacteria (See FIG. 5A) and wells containing only the colorimetric biosensor (See FIG. 5G) were also prepared. In order to induce the growth of MRSA and the color change of the biosensor accordingly, they were cultured at a temperature of 37° C., and each well plate was photographed at 0, 5, 6, 7, 9, 12, 13 and 15 hours to compare the color change.

Referring to FIG. 5, when only the colorimetric biosensor and MRSA were cultured, a color change (blue->red) appeared after 7 hours (t=7) (See FIG. 5A). On the other hand, in the absence of both antibiotics and bacteria, no color change was observed even after 15 hours (t=15) (See FIG. 5G). When 2 μg/mL ampicillin was co-incubated in a well containing MRSA and the colorimetric biosensor, a color change (blue->red) appeared after 9 hours (t=9) (See FIG. 5B), but when 4 μg/mL ampicillin was co-cultured, a color change (blue->red) appeared after 13 hours (t=13) (See FIG. 5C). It may be determined that MRSA has resistance to the antibiotic ampicillin, thereby causing a color change in the colorimetric biosensor when co-cultured. Next, when 0.25 μg/mL vancomycin was co-incubated in a well containing MRSA and the colorimetric biosensor, a color change (blue->red) appeared after 9 hours (t=9) (See FIG. 5D), but no color change was observed when 2 or 4 μg/mL vancomycin was co-cultured (See FIGS. 5E and 5F). When MRSA was incubated with the vancomycin antibiotic, it did not cause color change from 2 μg/mL, so it may be identified that it has susceptibility to the vancomycin antibiotic from the concentration of 2 μg/mL.

Therefore, it was confirmed that the colorimetric biosensor according to the present invention is a colorimetric biosensor for detecting microorganisms capable of real-time measurement and exhibiting excellent sensitivity or may be applied to a method for testing antibiotic susceptibility of microorganisms.

The above detailed description is illustrative of the present invention. Further, the above description represents and describes preferred examples of the present invention, and the present invention may be used in various other combinations, modifications and environments. That is, the scope of the concept of the invention disclosed in this specification may be changed or modified within the scope equivalent to the content of the disclosure and/or within the scope of skill or knowledge in the art. The written example describes the best state for implementing the technical idea of the present invention, and various changes required in the specific application field and use of the present invention may be made. Therefore, the detailed description of the present invention is only the disclosed examples and is not intended to limit the present invention. Further, the appended claims should be construed to cover other examples as well.

1. A colorimetric biosensor comprising:
a porous hydrogel structure formed of a hydrogel polymer and polydiacetylene; and
a microbial nutrient source.

2. The colorimetric biosensor of claim 1, wherein the microbial nutrient source is encapsulated inside the porous hydrogel structure.

3. The colorimetric biosensor of claim 1, wherein the microbial nutrient source is in the form of a shell surrounding the surface of the porous hydrogel structure.

4. The colorimetric biosensor of claim 1, wherein the hydrogel polymer includes at least one selected from the group consisting of alginate, agarose, chitosan, poly (ethylene glycol) diacrylate (PEG-DA), hydroxyethylene methacrylate (HEMA), polyvinyl alcohol (PVA) and polyacrylamide (PAM).

5. The colorimetric biosensor of claim 1, wherein the microbial nutrient source is at least one selected from the group consisting of Luria-Berani (LB) broth, LB agar broth, tryptic soy broth (TSB), lactobacilli MRS broth, R2A broth (MB-R2230), Mueller Hinton (M-H) broth, and a broth containing casein hydrolysate (casamino acid).

6. The colorimetric biosensor of claim 1, wherein the colorimetric biosensor detects a microorganism through color change.

7. The colorimetric biosensor of claim 1, wherein the microorganism is at least one selected from the group consisting of bacteria, fungus, alga, protozoan and isolated cells of metazoan.

8. A method for testing susceptibility to an antibiotic, the method comprising culturing a microorganism in the presence of the colorimetric biosensor according to claim 1 and the antibiotic.

9. The method of claim 8, wherein it is determined that the microorganism has susceptibility to the antibiotic when there is no colorimetric change of the colorimetric biosensor; and it is determined that the microorganism has resistance to the antibiotic when there is colorimetric change of the colorimetric biosensor.

10. A kit for testing susceptibility to an antibiotic, the kit comprising the colorimetric biosensor according to claim 1 and the antibiotic.

11. A method for preparing a colorimetric biosensor, the method comprising:
(a) obtaining a hydrogel precursor solution containing a polydiacetylene solution, a hydrogel polymer and a microbial nutrient source;
(b) dropping the hydrogel precursor solution into a calcium chloride solution to obtain a hydrogel structure; and
(c) irradiating ultraviolet rays having 200 to 300 nm wavelength to the hydrogel structure.

12. The method of claim 11, wherein the concentration of the hydrogel polymer in the hydrogel precursor solution is 1.0 to 15.0% (w/v)

13. The method of claim 11, wherein the concentration of the polydiacetylene solution is 0.5 to 5 mM.

14. The method of claim 11, wherein the concentration of the calcium chloride solution is 0.5 to 20.0% (w/v).

15. The method of claim 11, wherein the colorimetric biosensor comprises a porous hydrogel structure formed of a hydrogel polymer and polydiacetylene; and the microbial nutrient source, and
the microbial nutrient source is encapsulated inside the porous hydrogel structure, or the microbial nutrient source is in the form of a shell surrounding the surface of the porous hydrogel structure.
